(19) United States
(12) Patent Application Publication
(10) Pub. No.: US 2018/0000598 A1
(43) Pub. Date: Jan. 4, 2018

(54) HIP STEM

(71) Applicant: Imperial Innovations Limited, London (GB)

(72) Inventors: Andrew Amis, Middlesex (GB); Bidyut Pal, London (GB)

(21) Appl. No.: 15/543,402

(22) PCT Filed: Jan. 13, 2016
(86) PCT No.: PCT/GB2016/050078

§ 371 Date: Jul. 13, 2017

(30) Foreign Application Priority Data
Jan. 13, 2015 (GB) 1500481.5

(51) Int. Cl. A61F 2/36; A61F 2/30; A61F 2/28

(52) U.S. Cl. CPC A61F 2/3676; A61F 2/3672; A61F 2/30942; A61F 2002/30919; A61F 2002/30879; A61F 2002/30769; A61F 2002/30072; A61F 2002/30014; A61F 2002/30013; A61F 2002/2817; A61F 2002/3631; A61F 2002/3668; A61F 2002/30112

(57) ABSTRACT

A stem (100) for use in a joint prosthesis, such as a femoral stem for a hip joint prosthesis, the stem comprising: a solid central core (102); a proximal outer layer (127) disposed over a proximal portion (101a) of the central core, wherein the proximal outer layer comprises a set of longitudinal ribs (120), defining slots (130) there between; and a distal outer layer made of a deformable porous material disposed over a distal portion (101b) of the central core. The arrangement is such that the stem (100) can be made with a relatively large diameter yet without being excessively stiff, for cementless fixation in osteoporotic patients. The deformability of the distal outer layer also mitigates against the risk of intraoperative bone fractures.

### FIELD OF THE INVENTION

The present invention relates generally, but not exclusively, to prosthetic implants, particularly to implants used in joint replacement orthopaedic surgery, and more particularly to femoral stems used in hip replacement orthopaedic surgery.

### BACKGROUND TO THE INVENTION

The hip joint forms the connection between the femur and the pelvis. It consists of two main parts: a ball (femoral head) at the top of the femur (thighbone) that articulates into a round socket (acetabulum) in the pelvis (hipbone), resembling a ball-and-socket joint. The bone surfaces of the ball and socket have a smooth durable cover of articular cartilage that cushions the ends of the bones and enables them to articulate easily without damage. In a healthy hip, a smooth tissue called synovial membrane makes a small amount of fluid that lubricates and almost eliminates friction in the hip joint. The femur consists of a central cylindrical shaft, called the diaphysis and two wider and rounded ends, called epiphyses. Conical regions called the metaphysis connect the diaphysis with each epiphysis. Other than the femoral head, the proximal femur has a neck, a greater trochanter and a lesser trochanter.

The hip joint is a stable and multifunctional joint; the major function being load bearing. It also offers a range of motions during normal daily activities. The stability of the hip joint is provided by its relatively rigid ball and socket type configuration, its ligaments and by the large, strong muscles across it. In certain traumatic situations and arthritic conditions due to aging, the hip joint can be very debilitating; not only causing pain, but also limiting the ability to perform normal activities. Hip replacement often minimises or eliminates pain and improves mobility in patients.

In a typical total hip replacement procedure, the natural bearing of the joint is replaced by an artificial bearing and a structure to support the bearing. The damaged surface of the acetabular cavity is replaced by a mechanical component called an acetabular socket prosthesis. On the femoral side, a spherical head replaces the damaged femoral head. The spherical head is positioned and supported by a stem structure to make it articulate within the socket. The stem is a rod like structure, which consists of a neck, and a body that can be inserted within the femoral medullary canal, passing from metaphysis to the diaphysis but bypassing the greater trochanter. The medullary canal is prepared by a reaming/rasping/broaching operation after resecting the femoral head and neck. The neck of the stem transfers the joint load from the spherical head to the body of the stem which, in turn, transfers the load to the femur. The body of the stem has a distal tip which guides the body during insertion into the medullary canal. The implants, made of biocompatible materials, should be durable and capable of transferring the load to the femur effectively. The stem is often manufactured using Cobalt-Chrome, or Stainless steel or Titanium alloy.

The stem may be fixed to the femoral medullary canal in two ways; with or without using cement. In cemented fixation, the stem is inserted into the prepared medullary canal which is oversized and filled with bone cement. On insertion of the stem, the cement migrates to the internal trabecular structure to a certain extent to form a cement mantle. Additionally, a layer of cement is formed surrounding the body of the stem. The cement layer and mantle stabilises the stem within the canal. In cementless fixation, the stem is inserted in a press-fit manner into the medullary canal which is prepared undersized. The outer surface of the stem is typically coated with material that stimulates bone growth into the implant surface. The coating material may be of hydroxyapatite (HA), certain grade of bioglass (BG) or a porous surface layer in the form of a matrix of small metallic beads or a wire mesh. With recent improvements in uncemented fixation and developmental progress in prosthesis structure, this is increasingly being used in hip surgery in all groups of patients.

Both of the fixation methods have their own pros and cons. Cement fixation has certain well-known complications, such as thermal necrosis of the bone, increased risk of fat embolism and cardiopulmonary complications, particularly in elderly patients (Parvizi et al. 2004; Keisu et al. 2001). These difficulties could be partially avoided by the use of cementless components. Moreover, elderly patients, generally being more fragile mentally and physically than the younger patients, may benefit from a reduction of surgical time and hence reduced blood loss when using cementless fixation (Keisu et al. 2001).

Femoral internal morphology varies widely across the population; with gender, age and disease condition (osteoporosis) of the patients. Based on Dorr's classification system, the patterns of shape and structure of the femurs are broadly categorised as Type A, B, and C (Dorr et al. 1993). Type A (normal) bone has thick cortices seen on the anterior-posterior radiograph and a large posterior cortex seen on the lateral view. The medial and posterior cortices begin at the distal end of the lesser trochanter and are immediately quite thick. They create a narrow diaphyseal canal. Thick diaphyseal cortices also produce a funnel shape to the proximal femur. The cortical bone has distinct edges and the Roentgenographic appearance is dense. Type B (osteopenic) exhibits bone loss from the medial and especially posterior cortices. The most proximal portion of the posterior cortex is thinned or absent, which accounts for an increased width of the intramedullary canal. The posterior endosteal surface is irregular and may be scalloped. Type C (osteoporotic) bone has virtually lost the medial and posterior cortices. The anterior and posterior cortices may also be dramatically thinned so that the bone on the lateral radiograph has a fuzzy appearance. The intramedullary canal diameter is usually very wide, forming a stove-pipe like structure.

Major clinical complications of uncemented hip stems include unstable component fixation and osteolytic destruction of bone (Noble et al. 1995). Since primary stability of uncemented stems is determined by the frictional force between the implant and bone, achievement of proper fit of the implant into the bone during surgery is very important. However, sufficient stability cannot be obtained by conventional cementless femoral stem in osteoporotic (OP) femurs, probably because of poor geometric fit of the implant within the bone cavity (Noble et al. 1988; Noble et al. 1995; Lee et al. 2011; Mears et al. 2009). Conventional femoral stems, designed for a normal and healthy femur, do not match the shape of the proximal OP femoral canal.

Most designs of cementless femoral stems are offered in a number of standard sizes to accommodate different femoral canal diameters. In almost all systems, the femoral stem has a standard shape that is scaled to fit some standard dimension of the medullary canal, typically the reamed isthmus (Noble et al. 1995). Consequently, a conventional hip stem implanted in OP patients is prone to movement relative to the host bone while loaded during physiological activity, causing pain and discomfort. This leads to inhibition of use of the lower limb, deteriorating the quality of the patient's life.

Moreover, conventional stem designs can cause distal load transfer at the tip, the risk of intra-operative femoral fractures, and occurrence of thigh pain. In order to restore normal function and normal range of movements, it appears, therefore, that OP patients require a femoral stem with improved shape and with good bone ingrowth capabilities.

In recent times, efforts have been directed towards achieving stability of uncemented femoral stems by fit and fill of the normal femoral cavity. U.S. Pat. No. 4,813,963 (Hori et al.) discloses a stem having a configuration that reflects the anatomic contour of the medullary canal more accurately. The proximal portion of the stem, in transverse cross-section, has an asymmetric contour. In addition, the medial side is curvilinear in shape while the other sides have linear edges. Several patents disclose femoral stems for press-fit with biological fixation to the wall of the proximal metaphysis and intra-medullary canal (U.S. Pat. Nos. 5,004,476, 5,571,187). US patent application number 2004/0102854A1 presents a hip stem with a metallic core and proximal polymeric body with textured or porous surface aiming at improved osseointegration. U.S. Pat. Nos. 4,589,883, 4,738,681, and 5,776,204 disclose femoral stems having a twist in the proximal region for better fit and stability within the femoral canal. However, rotational motion of the stem induced by the twist may lead to the formation of a gap at the implant-bone interface. The twisting feature of the design also prevents the stem from sitting on the neck of the femur.

Attempts have also been made to increase stability of the implant by improving the bond between the implant and the bone. To provide more surface area for bone ingrowth, several depressions/recesses of different shape and sizes have been designed over the implant outer surface (U.S. Pat Nos. 4,430,761; 4,828,566; US patent application number 20080183298A1). Some of the earlier designs of the stem have tripartite differential porosity of the stem at different levels (US patent application number 20080183298A1).

In order to achieve a better geometric fit within the femoral cavity, the ideal design of an uncemented hip stem would be for it to have a tight proximal fill with anatomic medullary components, and a tight wedge fit with tapered prostheses in the diaphysis with coated stems. Since the OP femur has considerable cortical thinning in the medial, posterior and anterior walls, a press-fit stem within the thin-walled medullary canal may create high hoop stress in the surrounding cortical bone. This high hoop stress may make the thin cortical wall susceptible to fracture in osteoporotic patients (Abdul-Kadir et al. 2008). Risk of intra-operative peri-prosthetic femoral fracture in uncemented hip replacement is reported to range between 1.5-27.8% (Berend and Lombardi Jr., 2010). A small misalignment of the component within the canal may further aggravate the risk of fracture. Although this can be treated intra-operatively with circlage-wire, the peri-prosthetic fractures are associated with higher cost and increased operative time. If a fracture remains undetected intra-operatively, it may result in subsequent post-operative fracture requiring a revision surgery. Stem designs and instrumentations are reported to be associated with the risk of intra-operative femoral fracture (Berend et al. 2004; Berend and Lombardi Jr., 2010).

Clinical studies observed significant thigh pain in uncemented stems more commonly in osteoporotic (OP) and bone stock deficient femurs (Bezwada et al. 2004; Moreland and Marino, 2001; Engh et al. 1987). Thigh pain is commonly attributed to stem-bone micro-motions and stiffness mismatch between the implant and the bone. Some earlier designs provided distal slots/channels at the tip of the stem to reduce the stiffness and thereby occurrence of thigh pain. For example, U.S. Pat. Nos. 5,507,829 and 3,996,625 disclose designs of slots in the coronal plane of the femoral stem to reduce stiffness in one plane of bending. Noble et al. (1998) (U.S. Pat. No. 5,776,204) discloses an asymmetric stem design wherein the distal portion has a rotated internal slot for reducing stiffness in both coronal and sagittal planes. U.S. Pat. No. 5,152,799 discloses a stem design with two different zones of taper (proximal and distal) to avoid sudden changes in stress level in the bone at the distal tip of the stem.

Another commonly recognised problem of cementless press-fit hip stems is stress-shielding of the proximal femur. A press-fit hip-stem transfers the load more distally, where the stem contacts the endosteal surface of the medullary canal, shielding the proximal femur from load. The cause of stress-shielding is high stiffness of the conventional hip-stem. The stiff stem carries a major part of the joint load that was previously fully carried by the femur itself in the unoperated condition. Subsequent bone remodeling causes the proximal femoral bone to resorb, weakening the fit of the stem into the bone. Larger diameter stems are expected to cause more pronounced bone loss by stress shielding. This is because the axial rigidity of the implant increases directly with the cross-sectional area, and flexural rigidity increases directly with the area moment of inertia. Thus, a small increase in stem diameter can greatly increase its rigidity. Therefore, patients with large intra-medullary canals (osteoporotic) are more susceptible to stress-shielding when using a cementless stem.

In order to reduce the effect of stress-shielding related to the high stiffness of the implant, stems with reduced or varying stiffness along the length have been proposed. U.S. Pat. Nos. 5,152,799, 5,702,482, 5,509,935, 5,336,265, 5,007,931, 4,921,501, 4,808,186 disclose hip stems with slots and channels to reduce stiffness of the stem. However, these designs may not provide a sufficiently close fit or enough surface area for bone growth. Effort has been also directed to reduce stiffness by hollowing out the stem (U.S. Pat. Nos. 5,725,586; 5,316,550; 5,092,899). However, removing material central to the structure contributes little in reducing the stiffness of the implant. WO 2011/005126A1 discloses a hip implant system wherein the stem and the cup have a layered structure and contact between the bone and the implant occurs via a layer of plastic with suitable mechanical durability and elasticity close to the elasticity of the bone.

U.S. Pat. No. 6,887,278 discloses a prosthetic hip stem with varying stiffness along the length of the stem. The stem is comprised of an elongated core and multiple segments extending outwards from the core. The segments are spaced apart by circumferential grooves around the core. The length of the segments and the grooves, and the material for the core and the segments is chosen in a way that the stiffness of the stem reduces from the proximal end to the distal end. Thus, the stem will carry more load proximally than the distal part, reducing the effect of stress-shielding in the proximal part of the femur.

In order to eliminate the shearing stress concentration both in the distal end of the stem and in the proximal end, and to obtain physiological load transfer and high fit and fill within the cavity, the use of composite materials has been suggested for cementless hip stems. Carbon Fibre reinforced plastic has been suggested as an alternative to metal for designing such a hip stem (Bandoh et al. GB2432025A/AU2003280556A1/US2006184250A1/US2010312354A1). EP0570172A1 discloses a similar composite hip stem with a metallic core and braided fibre and thermoplastic resin shell, with varying stiffness along the length of the stem. U.S. Pat. No. 5,480,449 summarised earlier designs for composite hip stems and proposed a new composite design that was aimed at reducing modulus mismatch of the stems to the surrounding bone.

A few other uncemented stem designs (EP0623321A1/DE2839092C3/EP0093378B1/U.S. Pat. No. 4,608,053) have been proposed, having a central core and longitudinal ribs (with or without taper) protruding out radially from the core either in the proximal portion of the core or along the entire length of the core. They are claimed to achieve better primary and long-term stability (EP0623321A1), substantial filling of the spongy proximal part of the femur and hence improve load transfer proximally (EP0093378B1/ US4608053).

There are some clinical studies that report good results for osteoporotic/elderly patients undergoing hip surgery with some specific femoral stem designs (Meding et al. 2010; Kelly et al. 2007; Reitman et al. 2003; Keisu et al. 2001). Most of these designs include bi-planar tapered wedge-shaped geometry with circumferential porous coating at the proximal region. The tapered stems rely on initial three-point fixation followed by proximal bone ingrowth for continued stability. The points of fixation are achieved by the implant at two spaced points on the posterior surface of the stem, and an intermediate point in the anterior side of the stem. Consequently, such a stem may result in localised stress/strain-concentration around the point of support. This may lead to increased risk of intra-operative peri-prosthetic femoral fracture and/or post-operative localised pain in OP femurs. Furthermore, bone present in between the points of contact may become weakened before achievement of sufficient bone ingrowth.

US patent application numbers 20070219641A1/20100222893A1/20120136455A1/ (Dorr et al.), 20060276906A1 (Hoag et al.), 20080167723A1/20110166668A1 (Acker et al.) and U.S. Pat. No. 8,088,169B2 disclose femoral stem designs suitable for patients with certain types of anatomy, such as female patients and/or patients having osteoporosis.

Acker et al. proposed a set of hip stems of increasing nominal size, wherein the dimensions of the diaphyseal width increases substantially non-proportionately with corresponding increase in the metaphyseal width, offset and head-height dimensions. The femoral component consists of a metallic core (may be Cobalt-chromium or Titanium alloy) and a neck portion, a polymer matrix layer (PEEK-Polyetheretherketone) substantially (fully or partially selected part) covering the stem portion of the core, and porous metal layer (may be titanium fibres or other metal bead matrix) covering the polymer layer. The porous metal layer may extend distally along the full polymeric layer or up to a selected proximal part. The polymer layer connects the core and the porous layer and provides a reduced stiffness of the hip stem.

Dorr et al. disclose a similar femoral component in US patent application number 20100222893A1. These designs are commercially available as VerSys™ Epoch™ FullCoat femoral stem from Zimmer Inc. The large metaphyseal components add a section to the standard implants in the area of the medial curve to help fill the larger proximal-medial anatomies for a better stability. It has been claimed that this is expected to help the surgeon to accommodate patients with proximal/distal femoral canal mismatch, maintaining proximal fit and fill.

Only little attention has been paid to an uncemented stem suitable for osteoporotic femur anatomy. There is a need for an improved shape of the hip stem that would fit the osteoporotic medullary canal more accurately. Osteoporotic patients will require a large diameter stem. Therefore, there is a further need for lowering the stiffness of the stem to reduce the risk of intra-operative femoral fracture, effect of stress shielding and occurrence of thigh pain. In addition to that there is a need for improved coating that will provide more surface area for bone growth and enhance fixation by using osteoconductive material to encourage bone formation at the fixation interface.

### SUMMARY OF THE INVENTION

According to a first aspect of the invention, there is provided a stem for use in a joint prosthesis, the stem comprising a solid central core, a proximal outer layer disposed over a proximal portion of the central core, wherein the proximal outer layer comprises a set of longitudinal ribs, defining slots there between, and a distal outer layer made of a deformable porous material disposed over a distal portion of the central core.

This arrangement provides for a stem that is particularly suited for use in osteoporotic patients, especially for uncemented fixation, by virtue of the fact that it can have a relatively large outer diameter, yet not be excessively stiff. The deformability of the distal outer layer means that stress-concentration between the stem and the surrounding bone is reduced for improved load transfer and the risk of fracturing the bone during insertion of the stem is reduced. The porosity of the outer layer encourages bone in-growth. In certain embodiments, the distal outer layer need not be of a porous material, but must have the required deformability.

The portion of a stem closest to the joint is commonly known as the proximal portion and the portion of the stem furthest from the joint is commonly known as the distal portion.

The distal outer layer may be disposed over the distal third to half of the central core, and the proximal outer layer may be disposed over the corresponding proximal two thirds to half of the central core.

Alternatively, the distal outer layer may be disposed over the distal 10% to 50% of the central core, but preferably over the distal 20% to 40% of the central core.

Alternatively, the distal outer layer may be disposed over 10% to 50% of the length of the stem, but preferably over the distal 20% to 40% of the length of the stem.

A distal end of the stem may comprise a bullet-shaped or rounded tip. Such a profile to the tip helps to reduce the risk of damage to the patient's bone during insertion. The tip is preferably also made of a deformable porous material.

In one embodiment, the central core is tapered, narrowing towards the distal end. Such a tapering core would have varying stiffness along its length, reducing closer to the distal tip, reducing distal load transfer. The thickness of the distal outer layer may increase towards the distal end of the central core to ensure a constant outer diameter of the stem so that a good fit within the bone (e.g. a reamed medullary canal) is maintained.

The distal outer layer may be substantially cylindrical or trapezoidal in cross-section. A cylindrical outer profile would be for use with a correspondingly cylindrical reamed bone bore and is straightforward to manufacture and to prepare, whereas a trapezoidal profile would fit within a correspondingly trapezoidal bore, made using a trapezoidal rasp as known in the art, and would therefore resist rotation within the bore.

The ribs are typically disposed radially about the core. In one embodiment, the ribs are disposed radially about the core except for at a proximal, medial portion of the stem. In that embodiment, the medial portion of the most proximal region of the proximal outer layer may comprise a layer of porous material to encourage bone in-growth at the proximal region as well as at the distal tip.

One or more of a bone stimulating material, a bone replacement material, and a bioactive bone substitute material may be disposed within the slots. Again, the purpose of these features is to encourage osseointegration of the stem with the surrounding bone.

One or more of the longitudinal ribs may comprise a solid base portion and an outer face portion comprising a layer of porous material. The layer of porous material is typically deformable. The thickness, in a radial direction, of the solid base portion may vary along the length of the stem such that the solid base portion is thicker towards the proximal end of the stem and thinner towards the distal end of the stem. Additionally, the thickness, in a radial direction, of the outer face portion may vary along the length of the stem such that the outer face portion is thinner towards the proximal end of the stem and thicker towards the distal end of the stem. In this latter embodiment, the respective thicknesses of the base portion and the face portion may be balanced such that the stem has a constant outer diameter over at least the proximal outer layer.

The distal outer layer may also comprise a set of longitudinal ribs, defining slots there between. The ribs and slots on the distal outer layer are typically arranged radially about the core. One or more of a bone stimulating material, a bone replacement material, and a bioactive bone substitute material may be disposed within the slots on the distal outer layer. This is for the same purpose as for the ribs of the proximal outer layer.

The pore size of the porous material may vary along the length of the stem such that the pore size increases towards the distal end of the stem and decreases towards the proximal end of the stem.

Advantageously, the stiffness of the stem is reduced towards the distal end of the stem by varying the pore size in such way. This reduces the distal load transfer of the stem.

The pore size of the porous material may vary radially such that the pore size increases towards the centre of the stem and decreases towards the outer surface of the stem.

Advantageously, the deformability of the stem is increased towards the outer surface of the stem by varying the pore size in such way. This reduces stress-concentration between the stem and the surrounding bone further reducing load transfer and the risk of fracturing the bone during insertion of the stem. Additionally, stress shielding is reduced and, therefore, bone loss is reduced.

The density of the porous material may vary along the length of the stem such that the density increases towards the distal end of the stem and decreases towards the proximal end of the stem.

Advantageously, the stiffness of the stem is reduced towards the distal end of the stem by varying the density of the porous material in such way. This reduces the distal load transfer of the stem.

The density of the porous material may vary radially such that the porous material is more dense centrally and less dense towards the outer surface.

Advantageously, the deformability of the stem is increased towards the outer surface of the stem by varying the density of the porous material in such way. This reduces stress-concentration between the stem and the surrounding bone further reducing load transfer and the risk of fracturing the bone during insertion of the stem. Additionally, stress shielding is reduced and, therefore, bone loss is reduced.

The density and/or the pore size of the porous material can be tailored to give any desired stiffness along the length of the stem in combination with any desired deformability at the outer surface of the stem. As such, these parameters can be customised to meet the needs of a particular patient.

For example, where a patient is osteoporotic, the density and/or the pore size of the porous material can be configured to enable the use of a cementless stem, without a significant risk of bone damage as a good enough fit within a femoral medullary canal without the risk of bone damage caused by stress shielding.

At least one of the anterior, posterior and lateral sides of the most proximal region of the proximal outer layer may comprise a fin configured to prevent rotation of the stem when, in use, located within a femoral medullary canal and such that the cross-section of the stem is wider at the most proximal region of the proximal outer layer. This reduces the likelihood of rotational movement of the stem within a femoral medullary canal reducing the risk of bone damage.

The stem may further comprise a collar at a proximal end of the stem. This acts as a guide and prevents the stem from being inserted too far into the femoral medullary canal, reducing the risk of bone damage during insertion of the stem into the femoral medullary canal.

Preferably, a most distal surface of the collar is made of a porous material. This encourages bone ingrowth at the collar, helping to secure the stem in place.

The proximal outer layer may be substantially cylindrical or trapezoidal in cross-section, at least over a distal portion thereof.

The stem may be thicker towards the proximal end of the stem. Advantageously, the stiffness of the stem is reduced towards the distal end of the stem by reducing the thickness in such way. This reduces the distal load transfer of the stem.

The stem may further comprise means for attaching a head component to a proximal end of the stem.

The diameter of the core may be in the range between 8 and 14 mm.

The pore size of the porous material of the distal outer layer may range between 300 and 1000 microns.

The thickness of the distal outer layer may range between 3 mm and 8 mm.

The core may be made of a titanium alloy.

The core may be made of a porous material.

The core may be made of one or more of: trabecular or porous titanium, titanium alloy or tantalum.

The pore size of the porous material of the core may vary along the length of the stem such that the pore size increases towards the distal end of the stem and decreases towards the proximal end of the stem.

The pore size of the porous material of the core may vary radially such that the pore size increases towards the centre of the core and decreases towards the outer surface of the core.

The density of the porous material of the core may vary along the length of the stem such that the density increases towards the distal end of the stem and decreases towards the proximal end of the stem.

The density of the porous material of the core may vary radially such that the porous material of the core is more dense towards the centre of the core and less dense towards the outer surface of the core.

The distal outer layer may be made of one or more of: trabecular or porous titanium, titanium alloy or tantalum.

The diameter of the stem may be in the range between 14 mm and 28 mm.

The length of the stem may be in the range between 50 mm and 170 mm.

In an embodiment, the length of the stem may be in the range between 120 mm and 170 mm.

In an embodiment, the length of the stem may be in the range between 70 mm and 100 mm.

In an embodiment, the length of the stem is in the range between 50 mm and 70 mm.

Shallow stems, such as those where the length of the stem is in the range between 50 mm and 70 mm, may be used in younger patients or in patients who are having a first (primary) hip implant, allowing longer implants/stems to be used as revision implants/stems in the future when the primary implant needs replacing.

In this way, the bone below the shallow stem which has not been reamed or otherwise removed is undamaged and can be prepared to receive a new longer stem.

Moreover shallower implants are less invasive and cheaper to manufacture.

The different ranges are suitable to provide different ‘standard’ sized stems of differing designs, that are intended for fixations which are principally diaphyseal intramedullary, metaphyseal intramedullary, and within the femoral neck region, respectively

The stem may be a femoral stem for use in a hip joint prosthesis.

According to a second aspect of the invention, there is provided a method of manufacturing a stem as set forth in the first aspect, the method comprising the steps of:
using patient scan data to design a stem adapted to suit the patient's physiognomy; and
using additive manufacturing techniques to fabricate a stem according to the design.

### BRIEF DESCRIPTION OF DRAWINGS

Embodiments of the present invention will now be described, by way of example only, with reference to the accompanying drawings, in which:

FIG. 1 shows a hip stem with longitudinal ribs extending along the majority of the length of the stem and terminating at a bullet-shaped tip, according to one aspect of the invention;

FIG. 2a shows a longitudinal cross-section through the hip stem of FIG. 1 according to one embodiment, in which the longitudinal ribs have a porous outer layer and a solid base layer;

FIG. 2b corresponds to FIG. 2a, but shows an alternative embodiment in which the longitudinal ribs are porous throughout their depth;

FIG. 3a is a transverse cross-section viewed on A-A of FIG. 2a;

FIG. 3b is a transverse cross-section viewed on A-A of FIG. 2b;

FIG. 4a is a transverse cross-section viewed on B-B of FIG. 2a;

FIG. 4b is a transverse cross-section viewed on B-B of FIG. 2b;

FIG. 5 shows an alternative hip stem with a longer, rounded-end tip (shown transparent) and longitudinal ribs extending through a thickened proximal medial portion;

FIG. 6 is a lateral elevation of the hip stem of FIG. 5;

FIG. 7a is a transverse cross-section viewed on C-C of FIG. 6 according to an embodiment in which the longitudinal ribs have a porous outer layer and a solid base layer; and

FIG. 7b is a transverse cross-section viewed on C-C of FIG. 6, according to an alternative embodiment in which the longitudinal ribs are porous throughout their depth.

### DETAILED DESCRIPTION

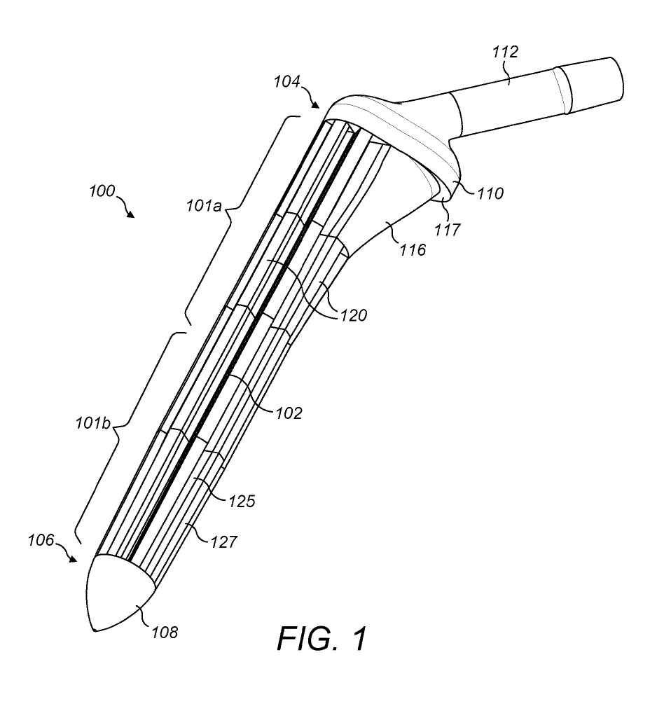

FIGS. 1 and 2a, 3a and 4b illustrate a first exemplary hip stem 100. The hip stem 100 comprises a central core 102 extending longitudinally within the stem from a proximal end 104 to a distal end 106. A tip 108 is located at the distal end 106 and a collar 110 is located at the proximal end 104. An attachment portion 112 extends from the collar 110 at the proximal end 104 of the stem 100 at an angle of approximately 45 degrees from a longitudinal axis of the stem in the medial direction, for the attachment thereto of a femoral head component (not shown), as known in the art. A thickened proximal medial portion 116 is located towards the proximal end 104, forming a buttress connecting the collar 110 with the main part of the stem for supporting load transfer to the proximal medial wall of the femur when the stem is located in situ within a patient's femoral canal. A lip 117 may be formed between a peripheral edge of the proximal end of the thickened proximal medial portion 116 and the underside of the collar 110, to prevent initial stem migration, as is known in the art.

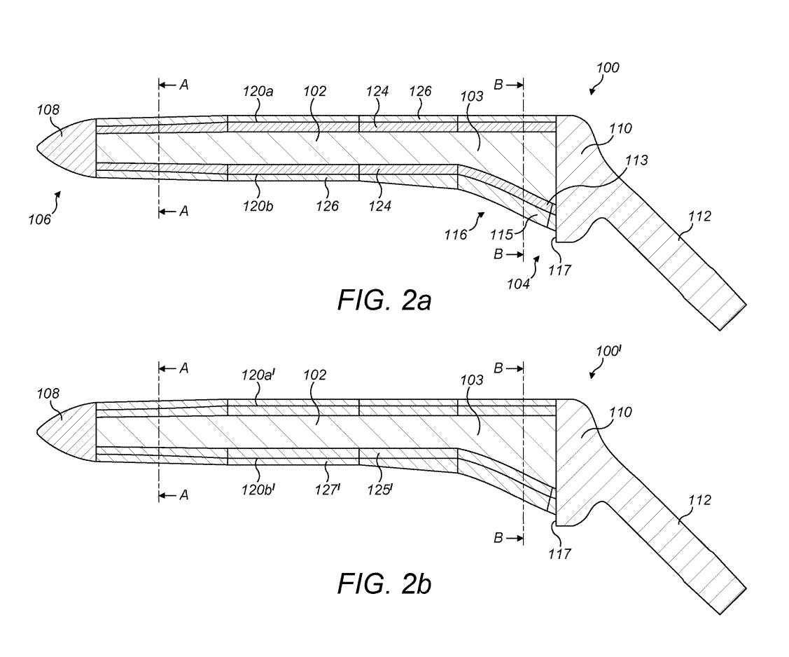

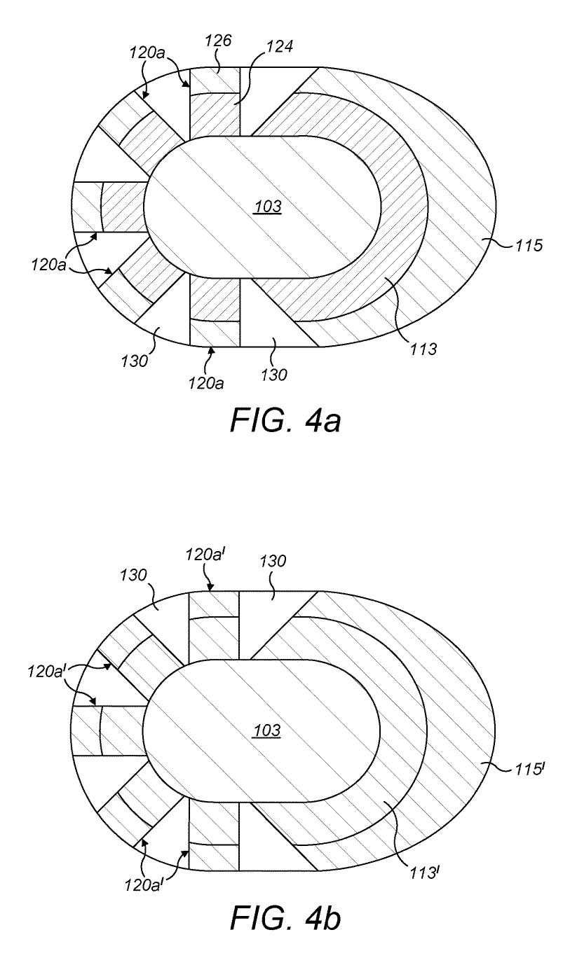

The core 102 is generally cylindrical; with a circular transverse cross-section, from the distal end 106 through to near the proximal end 104. Where the core extends through the thickened proximal medial portion 116, the core is itself thickened in a medial direction, forming an ovalised portion 103, as best seen in FIG. 4a, that increases in cross-sectional area towards the proximal end 104, as best seen in FIG. 2a.

The core 102 may be made of Ti or beta Ti alloy. Alternatively, the core 102 may be made of a porous material which may be deformable. Alternatively, the core 102 may be made of more of trabecular or porous titanium, titanium alloy or tantalum.

The collar 110 may be formed integrally with the core 102 or may be secured thereto in a separate step. In the latter case, the respective components may be formed of different materials, which may be selected to have particularly useful characteristics for their intended purpose.

A number of ribs 120 are disposed on the stem 100, extending radially from the core 102. On the lateral, anterior and posterior sides, the ribs 120a extend all the way from an underside of the collar 110 at the proximal end 104 to the tip 108 at the distal end 106. On the medial side, the ribs 120b in this embodiment run from the bottom end of the thickened proximal medial portion 116 to the tip 108.

As illustrated, there are eight ribs 120 in total, comprising 5 full-length ribs 120a disposed around the lateral, anterior and posterior sides, and 3 shorter ribs 120b on the medial side. The ribs 120 are disposed at equal angular intervals around the core 102. However, it will be appreciated that greater or fewer ribs 120 may be provided and that they need not be arranged with such rotational symmetry, nor project radially. Indeed, according to certain embodiments, it can be envisaged that asymmetrical arrangements of the ribs could be advantageous in order to better match the stiffness or bending strength of the device to the surrounding bone or the loads imposed.

As illustrated, the ribs 120 extend contiguously over both a proximal portion 101a and a distal portion 101b of the stem 100. However, in certain embodiments the ribs 120 would only extend along the proximal portion 101a, which may, for example, comprise the proximal-most half to two-thirds of the stem from beneath the collar 110 to the distal tip 108.

An inner, base portion 124 of each rib is, according to this embodiment, formed of a solid material, forming an intermediate layer 125 over the core 102. The intermediate layer 125 may be comprised of the same material as the core 102, which may be made of a light weight metal. The material of the core 102 and/or the intermediate layer 125 may be Ti or beta Ti alloy. The intermediate layer 125 may be fixed to the core 102 or it may be integrated with at least the proximal portion 101a of the core 102 such that the core 102 and the intermediate layer 125 form a single component.

An outer, face portion 126 of each rib comprises a porous or other low-stiffness material, such as a polymer: PEEK or polyethylene, by way of example; or a polymer composite: PEEK with granules of hydroxyl apatite embedded therein or polyethylene with granules of hydroxyl apatite embedded therein, by way of example. Likewise, the thickened proximal medial portion 116 comprises a solid inner portion 113 and a porous outer portion 115. The porous outer portions 126 and 115 together form a porous outer coating layer 127.

Such an intermediate layer 125 at the base of one or more of the ribs 120 would provide additional proximal stiffness and strength to that provided by the core 102. The intermediate layer 125 may be gradually tapered in a longitudinal direction such that the diameter thereof decreases towards the distal end 108, thus giving a gradual reduction of bending stiffness along the stem.

The core 102 and the inner portions 124 of the ribs 120, as well as the inner portion 113 of the thickened proximal medial portion 116, may be formed integrally in a single manufacturing step.

According to an alternative embodiment, as shown in FIGS. 2b, 3b and 4b, the stem 100′ is the same as the stem 100 described above with reference to FIGS. 2a, 3a and 4a (and like parts are referenced by common reference signs), except for the fact that the intermediate layer 125′ is not solid like the core 102, but is instead made of a porous material. Each rib 120′ thus comprises an inner, base portion 124′ that is formed of porous material and an outer, face portion 126′ that is also formed of porous material. In other words, each rib 120′ is formed of porous material throughout its depth, extending fully to the core 102. Likewise, the thickened proximal medial portion 116 comprises a porous inner portion 113′ and a porous outer portion 115′.

The inner and outer porous portions 124′, 113′ and 126′, 115′ (and therefore the intermediate and outer porous layers 125′, 127′) may be formed of the same porous material and be of the same density or may be formed of different porous materials and/or have different densities, preferentially with the least-stiff material at the outer surface. Where the inner and outer porous portions are formed of the same material, there is essentially no intermediate layer present, only the solid core 102 and a porous outer layer 127′.

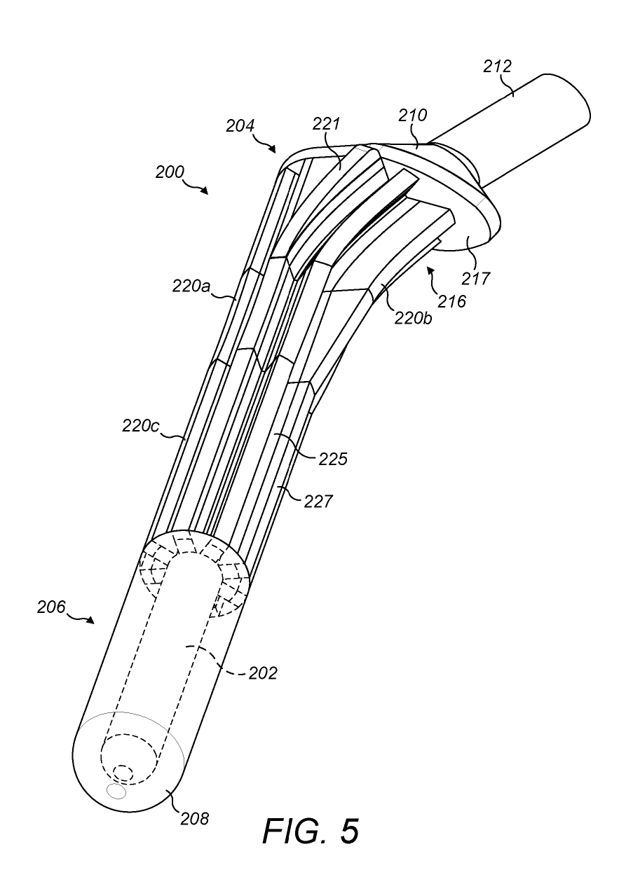

A further alternative embodiment of a stem 200 is shown in FIGS. 5 to 7b. The hip stem 200 comprises many of the same features as the stems 100, 100′ shown in FIGS. 1 to 4b (and like parts are referenced by common reference signs, albeit with the preceding ‘1’ replaced by a ‘2’), although the ribs 220 and associated slots 230 only extend along a proximal portion 201a of the stem, from the collar 210 to approximately half to two-thirds of the way to the distal end 206, with the distal portion 201b forming the remaining third to half of the length. Also, all of the ribs 220, including those on the medial side, extend right from the underside of the collar 210. The medial ribs 220b and the anterior and posterior ribs 220c each curve outwards in the medial direction at their proximal ends to form buttresses defining the thickened proximal medial portion 217.

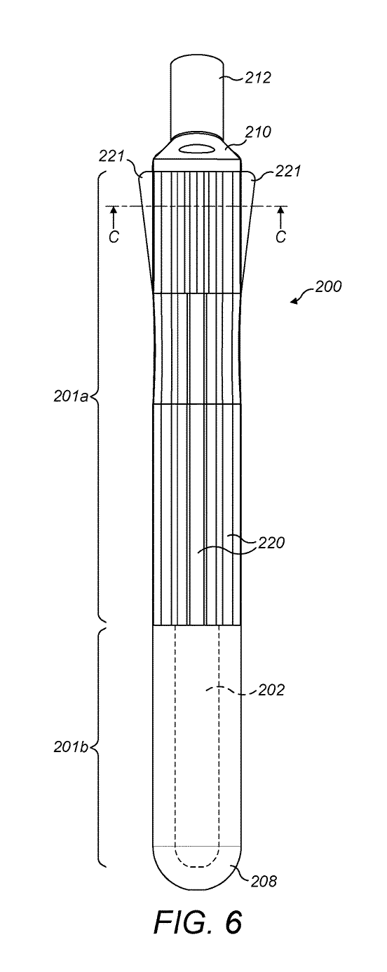

Each of the anterior and posterior ribs includes a fin portion 221 at its proximal end beneath the collar 210. As best seen in FIGS. 5 and 6, the fins 221 project outwardly beyond the profile of the collar 210 and the rest of the generally cylindrical profile of the stem to provide rotational stability of the stem when located in situ within the medullary canal. This is particularly advantageous for stems having a generally cylindrical cross section. A further fin (not shown) may project from the lateral side too.

Alternatively, rotational stability may be provided by having a non-cylindrical cross sectional profile at the proximal end 104, 204 of the stem. For example, a stem with a trapezoidal cross section would resist rotation in situ within a patient's medullary canal.

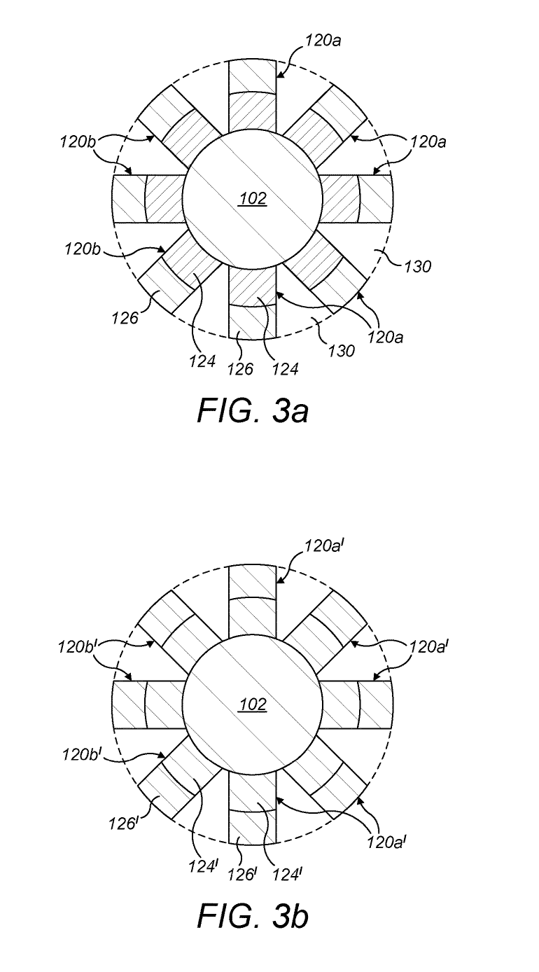

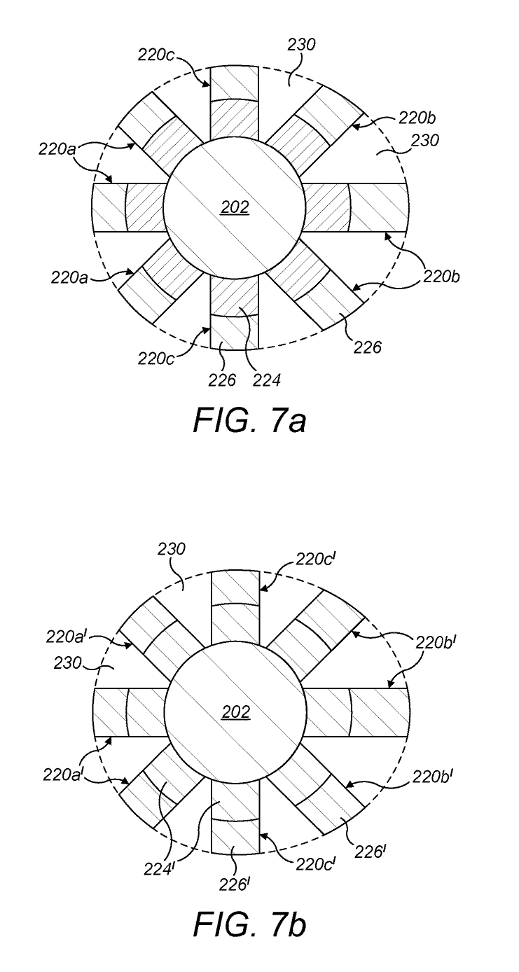

In a first arrangement, as shown in FIG. 7a, each of the ribs 220a, b, c comprises a solid, base portion 224 and an outer, face portion 226 of a porous material. In an alternative arrangement, as shown in FIG. 7b, each of the ribs 220a′, b′, c′ comprises a porous base portion 224′ as well as a porous outer, face portion 226′. The description above in connection with the constitution of the materials in the base and outer portions of the alternative embodiments set out in FIGS. 3a and 3b applies equally here.

The distal portion 201b of the stem 200 comprises a cylindrical sheath of porous material disposed around the distal portion of the core 202.

Hence, in the first arrangement of FIG. 7a, the stem 200 comprises a solid core 202, an intermediate layer 225 comprising base portions 224 of the ribs 220 over a proximal portion 201a of the stem, and an outer layer 227 comprising the porous outer, face portions 226 of the ribs 220 in the proximal portion 201a in conjunction with the cylindrical sheath of porous material disposed over the distal portion 201b. Likewise, in the arrangement of FIG. 7b, the stem 200 comprises a solid core 202, and an outer layer 227′ comprising the porous outer, face portions 226′ of the ribs 220′ in the proximal portion 201a in conjunction with the cylindrical sheath of porous material disposed around the distal portion 201b.

For all embodiments, the porous outer layer 127, 127′, 227 may comprise a coating layer which may, for example, be made of Trabecular Titanium (TT), porous titanium alloy, or Porous Tantalum (PT). Such a porous outer layer 127, 127′, 227 provides a low-modulus anchorage area to encourage bone in-growth from the surrounding bone cortex when the stem is inserted in a patient, and thus to secure the stem in situ.

The pore size of the porous material may range between 300 to 1000 microns, preferably between 300 to 600 microns, or more preferably between 300 to 500 microns. The density and/or pore size of the porous material may vary along the length of the stem, being more dense and/or porous (i.e. having less pores and/or having larger pore sizes) distally. It may also vary radially, being denser and/or less porous near to the central core and least dense and/or more porous at the outer surface, for example.

At least the porous outer layer of the distal portion 101b, 201b is deformable such that it reduces stress-concentration, distal load transfer and risk of intraoperative and postoperative femoral fracture.

The central core 202 may taper along the length of the prosthesis, such that it has a smaller diameter at the distal tip. It may also not extend the full length of the stem, leaving only a porous structure at the distal tip in zone 201b.

The ribs 120, 220 may range from 2 to 5 mm in circumferential width and may range from 3 mm to 8 mm in radial height. Accordingly, the outer layer 127, 127′, 227 may have a thickness in the range of 3 to 8 mm or, preferably, 4 to 7 mm. The outer diameter of the porous outer layer 127, 127′, 227 (and thus of the stem 100, 100′, 200) may be in the range of 16 to 28 mm, which may be the same size as or a bit smaller than that of the diameter of a femoral canal into which it is to be inserted. The stems 100,100′, 200 may be designed such that they are undersized, i.e. so that there is no contact between the stem and the surrounding bone, thus avoiding initial distal load transfer from the stem to the bone.

The diameter of the core 102, 202 may be in the range of 8 to 14 mm, but this is dependent upon the overall size of the stem 100, 100′ and 200. The core 102, 202 may be tapered such that it is thicker at the proximal end 104, 204 of the stem 100 and 200 and thinner at the distal end.

Slots 130, 230 are formed in the spaces between the ribs 120, 220. The slots may be 4 to 7 mm deep (from the height of the ribs 120, 220 to the outer edge of the core 102, 202). Depending on the arrangement of the ribs 120, 220, the slots may have an angular spacing of, for example, 30 to 45 degrees.

The slots 130, 230 reduce the stiffness of the proximal portion 101a, 201a of the stem 100, 100′, 200 (in comparison to a stem having the same diameter but being solid, rather than a having a rib/slot arrangement). Where the ribs/slots extend over the distal portion 101b too, the stiffness of that distal portion 101b of the stem 100, 100′ is also reduced. Moreover, the slots 130, 230 create spaces in which a special coating or filling can be placed; for example, a bone stimulating/replacement material which provides a high rate of osseointegration, even for osteoporotic or poor quality bone. One or more of any suitable known bioactive bone substitute materials may also be placed in the longitudinal slots 130, 230. Such a filling would encourage bone formation around the stem 100, 100′, 200. In certain embodiments, some or all of the slots 130, 230 may be left empty.

In one embodiment (not shown), the distal portion 201b is as described above, but rather than the ribs 220 over the proximal portion 201a having a porous outer face 226, they are solid, typically being formed of the same material as the central core 202.

Common to all embodiments is the combination of a solid core and at least a distal-most portion that is formed of a deformable porous material.

The distal tip 108 of the embodiments of FIGS. 1 to 4b is bullet shaped, whereas the distal tip 208 of the embodiments of FIGS. 5 to 7b is rounded. It will be understood, however, that any of the stems herein described may comprise either a bullet-shaped or a rounded tip.

The distal tip 108, 208 is preferably made of a porous material; most preferably the same as that of the porous outer layer 127, 127′, 227. Likewise, the underside of the collar 110, 210 including the lip 117, 217 if present, may be formed of a similar porous material to encourage bone in-growth for anchoring the stem in situ, mitigating against the usual scenario where bone-collar contact is lost due to bone resorption post-surgery.

The stems 100, 100′, 200 are typically symmetrical in the anterior/posterior plane, allowing for use on either the left or right side of the body, thereby avoiding the need for surgeons to have access to separate inventories for left and right side operations. However, it can be envisaged that stems could be designed asymmetrically for specific left or right side use. The proximal portion 101a, 201a of the stem may be curved to match medullary canal geometry, as known in the art.

It will be understood that any number of ribs 120, 220 may be provided around any of the central cores 102, 202 described herein, although, preferably, there are 8 to 12 ribs.

Where this document refers to porous material, it is primarily used to encourage bone ingrowth and also to provide reduced material and structural stiffness, but the parts described above as being porous may also be made of other low-stiffness material or composite such as a polymer, that may not be porous, yet still have the desired structural effect of reduced stiffness when compared against a solid cylindrical component. Examples of such materials include the polymers PEEK and polyethylene, as well as the polymer composites PEEK with granules of hydroxyl apatite in it and polyethylene with granules of hydroxyl apatite in it.

The stems 100, 100′, 200 shown in the Figures have a substantially cylindrical geometry below their thickened proximal medial portions 116 and 216. The diameter of the stems 100, 100′, 200 may range from 14 to 28 mm. Such a cylindrical geometry facilitates easy insertion of the stem into a prepared femoral canal, thus reducing the risk of intra-operative femoral fracture. Where the core 102, 202 is tapered, in order for the stem to have a cylindrical outer profile, the intermediate and outer layers will be correspondingly tapered in the opposite sense: i.e. thicker at the distal end 106, 206. This may be achieved by having the base 124, 224 with a constant depth and the outer face 126, 226 getting thicker (in depth) towards the distal end, or by having the outer face 126, 226 of a constant depth and the base 124, 224 getting thicker (in depth) towards the distal end, or a combination of the two.

Alternatively, the stems could also have a tapered geometry below their thickened proximal medial portions 116, 216, such that the diameters of the stems decrease towards the distal end 106, 206. Such a profile would also facilitate easy insertion of the stem into a prepared femoral canal.

The overall shape of the proximal part of the stems 100 and 200 may be configured to fully fill the femoral medullary canal. This may be achieved by providing a stem 100, 100′, 200 having a geometry which matches, for example, either a ‘normal’ or an osteoporotic femoral canal geometry. By way of example, the geometry may be adapted to a smaller intramedullary canal diameter and to a different pattern of taper along its length.

Multiple sizes of stems 100, 100′, 200 may be provided as standard to fit the variable sizes of different patients. For example, the stems may be available in a range of ‘small’, ‘medium’ and ‘large’ sizes.

The stems 100, 100′, 200 may be shortened so that they fill only a small metaphyseal zone of the proximal end of the femur, or even fit only into the neck of the femur.

The stem 100,100′, 200 may be used as a conventional stem, in which case it would be in the range of 120 mm to 170 mm long. The stem 100,100′, 200 may instead be used as short-stem prostheses, in which case it may be in the range of 70 mm to 100 mm long. According to a further embodiment, the stem 100, 100′, 200 may be used as a much more localised hip femoral component, which places a short stem coaxial with the neck of the femur, in which case it would be in the range of 50 mm to 70 mm long.

The porous material of the stems may have variable structure, such that it is stronger/stiffer in some zones, and softer in others, depending on the load transfer requirements. For example, the distal tip may be relatively soft/deformable, to reduce load/stress concentration here. This type of micro-structural adaptation may be built using ‘rapid prototyping’ methods, in material such as titanium alloy.

The bone cavity into which the stem 100, 100′, 200 is to be inserted may be prepared such that it is circular in cross-section, by a drilling type operation.

Alternatively, a more complex bone cavity could be made by a broaching tool, such that the cavity fits to any ribs and/or slots present in the stem 100, 100′, 200, or to match the trapezoidal geometry where included.

According to certain embodiments, the stem could be designed and manufactured for patient-specific application. As known in the art, a scan of the patient can be made (e.g. a CT scan) to collate scan data that can then be used in a planning/design phase to design a stem to suit a particular patient-specific need, optionally taking into account a surgeon's expert input. For example, the scan data can reveal the local joint topography and the surgeon can identify which parts of the bone should be resected during a joint replacement or repair operation. Optionally, the data can be used to match stem stiffness as closely as possible to the surrounding joint physiognomy. Using those inputs, a patient-specific stem can be designed to match the existing joint topography and to replace those portions of bone that are to be resected. Once designed, the stem can be manufactured using known manufacturing techniques, such as rapid prototyping or additive manufacturing, which are particularly suited to providing stems having variable structure throughout, such as having varying porosity to create areas with a range of stiffness, in order to optimise load transfer and reduce stress-shielding and stress concentrations. Software may be provided to assist in the design and operational planning phases.

Although the invention has been described in the context of a femoral stem for a hip joint prosthesis, the skilled person will appreciate that the teaching herein may instead be applied mutatis mutandis to other joint prostheses, such as either or both of a tibial or femoral stem for a knee joint prosthesis, or for the proximal humerus in shoulder replacement.

1. A stem for use in a joint prosthesis, the stem comprising:
a solid central core;
a proximal outer layer disposed over a proximal portion of the central core, wherein the proximal outer layer comprises a set of longitudinal ribs, defining slots there between; and
a distal outer layer made of a deformable porous material disposed over a distal portion of the central core.

2. The stem of claim 1, wherein the distal outer layer is disposed over the distal third to half of the central core, and the proximal outer layer is disposed over the corresponding proximal two thirds to half of the central core.

3. The stem of claim 1, wherein a distal end of the stem comprises a bullet-shaped or rounded tip.

4. The stem of claim 3, wherein the tip is also made of a deformable porous material.

5. The stem of claim 1, wherein the central core is tapered, narrowing towards the distal end.

6. The stem of claim 5, wherein the thickness of the distal outer layer increases towards the distal end of the central core.

7. The stem of claim 1, wherein the distal outer layer is substantially cylindrical or trapezoidal in cross-section.

8. The stem of claim 1, wherein the ribs are disposed radially about the core.

9. The stem of claim 1, wherein the ribs are disposed radially about the core except for at a proximal, medial portion of the stem.

10. The stem of claim 9, wherein the medial portion of the most proximal region of the proximal outer layer comprises a layer of porous material.

11. The stem of claim 1, wherein one or more of a bone stimulating material, a bone replacement material, and a bioactive bone substitute material is disposed within the slots.

12. The stem of claim 1, wherein one or more of the longitudinal ribs comprises a solid base portion and an outer face portion comprising a layer of porous material.

13. The stem of claim 12, wherein the layer of porous material is deformable.

14. The stem of claim 12, wherein the thickness, in a radial direction, of the solid base portion varies along the length of the stem such that the solid base portion is thicker towards the proximal end of the stem and thinner towards the distal end of the stem.

15. The stem of claim 14, wherein the thickness, in a radial direction, of the outer face portion varies along the length of the stem such that the outer face portion is thinner towards the proximal end of the stem and thicker towards the distal end of the stem.

16. The stem of claim 1, wherein the distal outer layer comprises a set of longitudinal ribs, defining slots there between.

17. The stem of claim 16, wherein the ribs and slots on the distal outer layer are arranged radially about the core.

18. The stem of claim 16, wherein one or more of a bone stimulating material, a bone replacement material, and a bioactive bone substitute material is disposed within the slots on the distal outer layer.

19. The stem of claim 1, wherein the pore size of the porous material varies along the length of the stem such that the pore size increases towards the distal end of the stem and decreases towards the proximal end of the stem.

20. The stem of claim 1, wherein the pore size of the porous material varies radially such that the pore size increases towards the centre of the stem and decreases towards the outer surface of the stem.

21-46. (canceled)
